(19) United States
(12) Patent Application Publication
(10) Pub. No.: US 2008/0255520 A1
(43) Pub. Date: Oct. 16, 2008

(54) Multiple injection syringe holder

(76) Inventor: Thomas D. Henderson, Lagrange, OH (US)

Correspondence Address: BARBARA E. ARNDT, PH.D., 26 NANTUCKET ROW, ROCKY RIVER, OH, 44116, US

(21) Appl. No.: 12/080,882

(22) Filed: Apr. 7, 2008

Related U.S. Application Data
(60) Provisional application No. 60/922,778, filed on Apr. 11, 2007.

(51) Int. Cl. A61M 5/19

(52) U.S. Cl. 604/191

(57) ABSTRACT

The invention provides a lightweight, inexpensive syringe holder for substantially simultaneous multiple injections such as, but not limited to, intramuscular injections for immunizations. The syringe holder can be molded as a single unit, and can be disposable or reusable, and easily cleanable and/or sterilizable, as desired. The syringe holder can securely accommodate two or more syringes, and two or more substantially simultaneous injections can be performed easily by the administrator using only one hand. Thus, it is possible for the administrator to use two hands, and perform four or more injections that are substantially simultaneous, thus reducing pain, stress and trauma.

This application claims the benefit of U.S. Provisional Application, Ser. No. 60/922,778, filed Apr. 11, 2007.

### BACKGROUND OF THE INVENTION

It has long been recognized by the medical community that the multiple immunization inoculations required for children, as well as booster inoculations required for adults, often necessitate multiple successive single injections. These multiple injections are not only time-consuming for the person administering the injections, but also can result in pain, stress and trauma to both the patient and the administrator.

In response to this problem, attempts have been made to provide a device that holds multiple syringes in order to produce simultaneous injections. As discussed in U.S. Pat. No. 3,552,394, there is a well-known phenomenon of the two-point threshold of pain or pressure, defined as the distance at which the surface pain sensors of the human body cannot distinguish between single and multiple locations of pain. In other words, when applied to certain locations on the body, a person cannot distinguish between the applications of two or more needles when these needles are disposed comparably close with one another and, therefore, the person experiences a single sensation of pain. The two-point sensation of pain distance varies over different parts of the body. In some parts of the body, for example, the thigh and upper arm, the two-point threshold of pain may involve a distance of up to about 68 mm.

However, injection devices designed to take advantage of this phenomenon heretofore have been relatively complicated to produce, and some require sterilization between uses. Therefore, there is still a need for a device that can take advantage of the two-point threshold of pain or pressure by providing substantially simultaneous multiple injections, especially for use in providing pediatric and adult immunization inoculations.

### SUMMARY OF THE INVENTION

The invention provides a lightweight, inexpensive syringe holder for substantially simultaneous multiple injections such as, but not limited to, intramuscular injections for immunizations. As defined herein, “substantially simultaneous injections” means that two or more injections can be performed by the administrator in a motion that enables the needles attached to two or more syringes to penetrate the skin and/or muscle of a person at essentially the same time, such that the person experiences a single sensation of pain.

A syringe holder according to the invention has an “open” design that does not require that the syringe barrels be enclosed in the holder or to be an integral part of the holder. The syringe holder can be molded as a single unit, and can be disposable or reusable, and easily cleanable and/or sterilizable, as desired. The syringe holder can securely accommodate two or more syringes, and two or more substantially simultaneous injections can be performed easily by the administrator using only one hand. Thus, it is possible for the administrator to use two hands, and perform four or more injections that are substantially simultaneous, thus reducing pain, stress and trauma.

In particular, the invention provides a syringe holder for multiple injections, that comprises (a) a substantially flat housing comprising a top edge, a bottom edge, two side edges and a front surface, wherein the housing has a width sufficient to accommodate two or more substantially parallel syringes spaced apart from each other in a plane parallel to the front surface, wherein each syringe comprises a syringe barrel, a syringe plunger and an injection needle; (b) a first set of substantially horizontal adjacent ledges contiguous with the front surface of the housing, wherein the first set of adjacent ledges is linearly separated by a set of first spaces, each of the first spaces being of sufficient width to accommodate the syringe barrel but not of sufficient width to accommodate a syringe lip contiguous with the syringe barrel, and wherein the first set of adjacent ledges is optionally contiguous with at least a portion of the top edge of the housing; (c) a plurality of flexible stabilizing devices contiguous with the front surface of the housing and aligned with the set of first spaces for releasably securing the two or more substantially parallel syringes to the housing, wherein the injection needles comprise needle tips extending substantially an equal distance below the bottom edge of the housing, and wherein at least two of the injection needles are spaced apart from each other at a distance that is at or within a two-point threshold of pain or pressure distance; (d) a depressible push apparatus comprising a plunger shaft and an associated cross piece, wherein the cross piece is configured to be in alignment with the two or more syringe plungers for substantially simultaneous depression of the plungers; and (e) a plunger shaft stabilizing device contiguous with the front surface of the housing for slideably engaging the plunger shaft.

In other non-limiting arrangements of the syringe holder, each of the first set of adjacent ledges can further comprise a ledge wall for containing the syringe lip within a first space between two adjacent ledges; and/or the top edge of the housing can comprise a contiguous lateral extension that comprises a grip surface; and/or the housing can further comprise a side wall contiguous with the housing at a side edge and the side wall can optionally comprise a grip surface. Further, the side wall can comprise a contiguous lateral extension that comprises a grip surface. In further non-limiting arrangements of the syringe holder, the housing can further comprise a bottom wall contiguous with the bottom edge of the housing. The bottom wall can comprise a set of second spaces, each of the second spaces being in direct opposition to a corresponding first space of the first set of substantially horizontal adjacent ledges, each of the second spaces being of sufficient width to accommodate a syringe needle hub but not of sufficient width to accommodate the syringe barrel.

In each of the arrangements of the syringe holder, the cross piece associated with the plunger shaft can be contiguous with the plunger shaft and, further, can comprise a first lateral cross piece section and a second lateral cross piece section, wherein the cross piece sections are offset from each other along the plunger shaft and each of the cross piece sections can be contiguous with the plunger shaft. This type of arrangement can be used for syringes having different sizes.

In a particularly useful arrangement, all of the components of the syringe holder, with the exception of the depressible push apparatus, can comprise a single molded unit. That is, the syringe holder can comprise one piece that is inexpensive to produce and lightweight to use.

### BRIEF DESCRIPTION OF THE DRAWINGS

FIG. 1 illustrates a syringe holder according to the invention.

FIG. 2 illustrates another arrangement of the syringe holder of FIG. 1 containing two syringes.

FIG. 3 illustrates elements of a syringe to be contained in the syringe holder.

FIG. 4 is a top elevation view of the horizontal ledges upon which the syringe lips can rest. Also illustrated is an opening in one of the ledges for passage of a depressible push apparatus.

FIG. 5 illustrates the position of the depressible push apparatus in relation to the syringe plungers of the filled syringes.

FIG. 6 illustrates the position of the depressible push apparatus after substantially simultaneous expulsion of the syringe contents.

FIGS. 7 and 8 illustrate a top view of an arrangement of the syringe holder illustrating the rotationability of the depressible push apparatus.

FIGS. 9 and 10 illustrate alternative stabilizing devices for the depressible push apparatus.

FIG. 11 illustrates another arrangement of the syringe holder, comprising a bottom wall.

FIG. 12 illustrates the arrangement of FIG. 11, with the presence of two syringes.

FIG. 13 illustrates an arrangement of the syringe holder for use with syringes of different sizes.

FIG. 14 illustrates the position of the depressible push apparatus in relation to the syringe plungers of filled syringes of different sizes.

FIG. 15 illustrates the position of the depressible push apparatus after substantially simultaneous expulsion of the syringe contents.

FIG. 16 illustrates an arrangement of the syringe holder having a top wall.

### DETAILED DESCRIPTION OF THE INVENTION

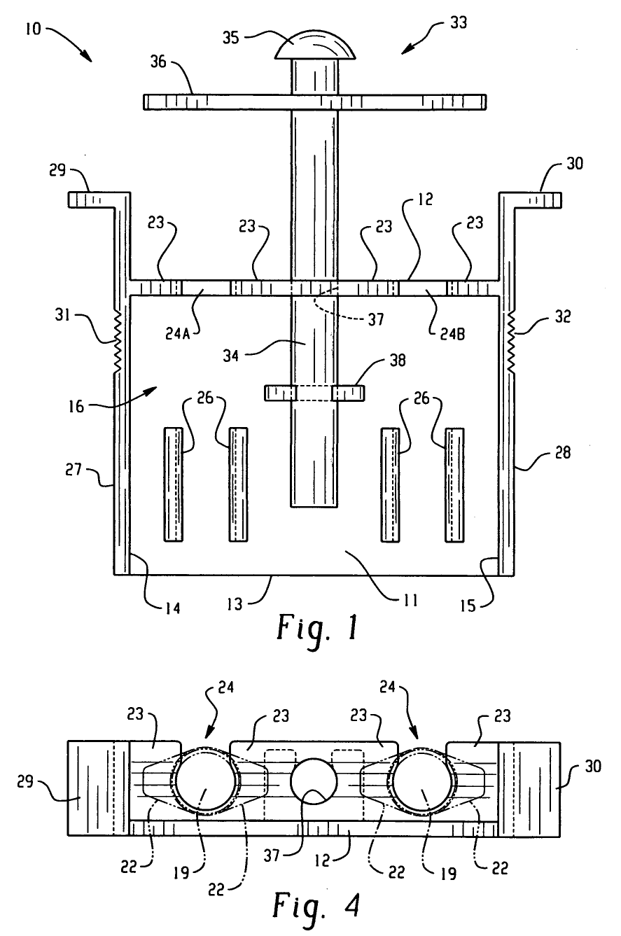

Several arrangements of the syringe holder according to the invention are illustrated in FIGS. 1 through 16. In particular, various arrangements of the syringe holder are illustrated and these arrangements, as they respectively correspond with their uses for multiple injections, are also illustrated. For convenience, many of the respective elements of the syringe holder and syringes are given the same descriptive numbers in the arrangements shown.

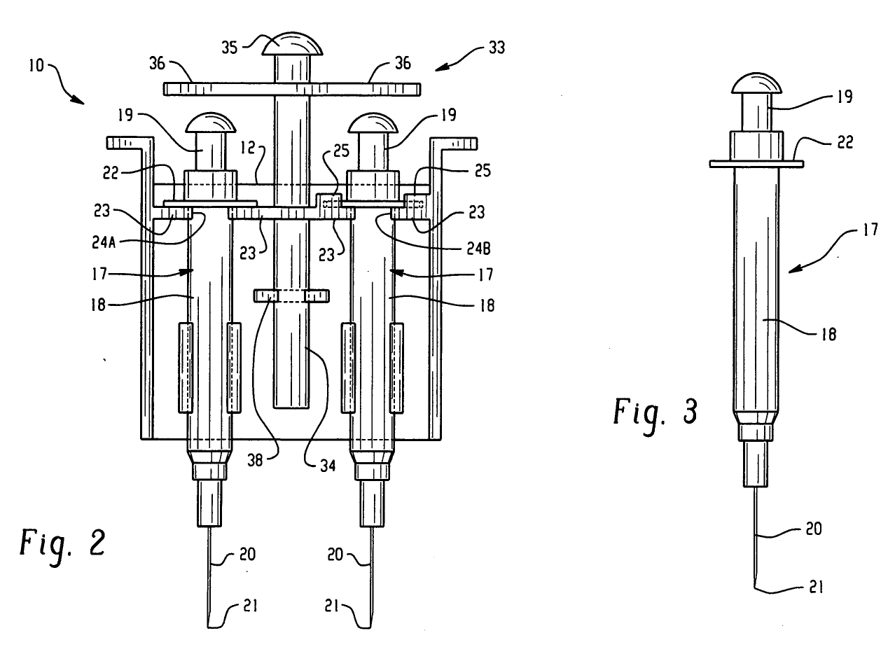

In the arrangement illustrated in FIG. 1, the syringe holder 10 comprises a substantially flat housing 11, having a top edge 12, a bottom edge 13, two side edges 14, 15, and a front surface 16. The housing 11 has a width sufficient to accommodate two or more substantially parallel syringes 17 spaced apart from each other in a plane parallel to the front surface 16, as illustrated in FIG. 2. Each of the syringes 17 has a syringe barrel 18, a syringe plunger 19, and an injection needle 20 having a needle tip 21. Each syringe 17 also has a syringe lip 22 that is contiguous with the syringe barrel 18. The syringes illustrated in FIGS. 2 and 3 and show the syringe plunger 19 partially extended to accommodate syringe contents for injection (not shown).

The housing 11 further has a set of substantially horizontal adjacent ledges 23 linearly separated by a set of spaces 24A, 24B. The set of adjacent ledges 23 are contiguous with the front surface 16 of the housing 11 and can be positioned at or below the top edge 12, as shown in FIGS. 1 and 2, respectively, as well as in other illustrated arrangements herein. The width of each of the spaces 24A, 24B between the ledges is sufficient to accommodate the syringe barrel 18 but not of sufficient width to accommodate the syringe lip 22. Thus, each of the syringe lips 22 can rest on two adjacent ledges 23, and contain the associated syringe barrel 18 within a space 24A, 24B. As illustrated in FIG. 2, one or more of the set of ledges 23 can optionally have a vertically extending ledge wall 25 that acts as a barrier to contain a syringe lip 22 within a space 24A, 24B.

The housing 11 further has flexible stabilizing devices 26 contiguous with the front surface 16 and aligned with the set of spaces 24A, 24B for releasably securing each of the syringes 17 to the housing 11. The stabilizing devices 26 can be any type of fastener such as, but not limited to, flexible clips, tubes, rings and the like. Although only one pair of stabilizing devices 26 is shown for each syringe 17 in FIGS. 1 and 2, it is recognized that the housing may contain any number of any type of stabilizing devices per syringe, as necessary.

The syringe holder 10 further can have one or two side walls 27, 28 being respectively contiguous with the housing 11 at a side edge 14, 15. In the arrangement of the syringe holder illustrated in FIG. 1, one or both of the side walls 27, 28 can extend beyond the top edge 12 to provide one or more grip surfaces 29, 30 for ease of handling by the administrator of the injections. For example the grip surface(s) 29, 30 can be grasped by the administrator using one or more fingers, while leaving a thumb or palm free to depress a push apparatus 33 (described below). The grip surface(s) 29, 30 can comprise a grooved or roughened surface. In another arrangement, the grip surface(s) 29, 30 can be replaced with or supplemented by one or more grip surfaces 31, 32 on the surface of one or more of the side walls 27, 28, such as by a grooved or roughened surface on one or more of the side walls.

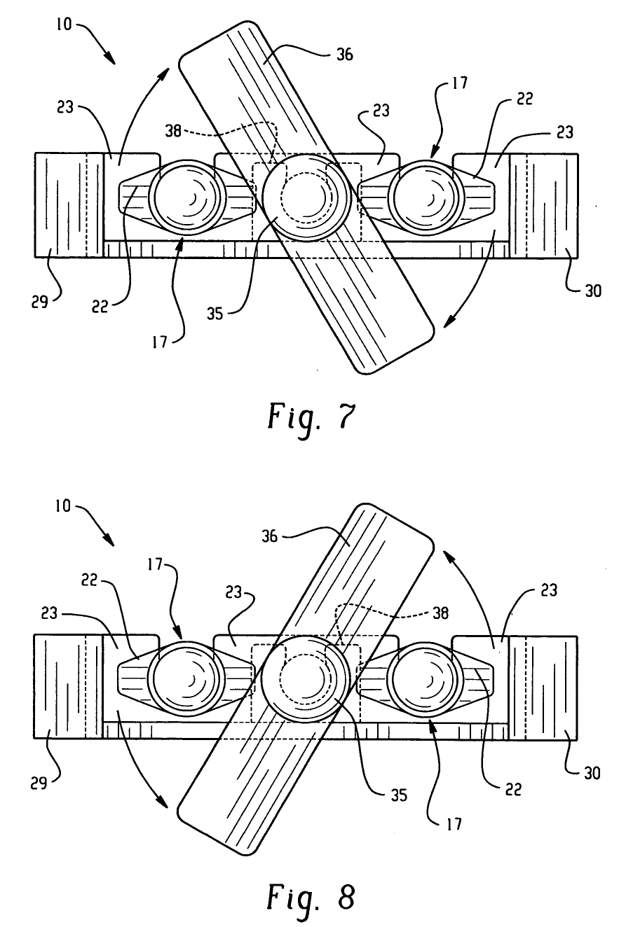

The syringe holder further has a depressible push apparatus 33. FIGS. 1 and 2 illustrate a suitable, but not limiting, push apparatus 33 that comprises a plunger shaft 34 and an optional head piece 35 for conveniently accommodating the thumb, palm or finger of the injection administrator. In this arrangement, the push apparatus 33 comprises a cross piece 36 preferably contiguous with the plunger shaft 34, that has a width sufficient to overlap two or more syringe plungers 19 associated with the two or more syringe barrels 18, such as those illustrated in FIG. 2. As illustrated in FIG. 4, for stabilization of the push apparatus 33, an opening 37 is provided in at least one of the set of ledges 23, the opening 37 having a size and shape sufficient to accommodate slideable passage of the plunger shaft 34. Further, one or more plunger shaft stabilizing devices 38 are provided that are contiguous with the front surface 16 of the housing 11 for slideably engaging the plunger shaft 34. The stabilizing device 38 is not limited in shape or size. For example, in the arrangement shown in FIG. 1, the stabilizing device 38 is a flexible clip. However, other non-limiting arrangements, such as a closed tube 38A or an open tube 38B illustrated in FIGS. 7 and 8, can be employed. There is no limitation on the shape of the plunger shaft 34 or the head piece 35. That is, the plunger shaft can have an angular, squared or rectangular shape, or can be ovoid or tubular in shape. The shape of the stabilizing device 38 and of the opening 37 in the set of ledges 23 is appropriate to the shape of the plunger shaft 34 to allow slideable and/or rotational passage of the plunger shaft 34.

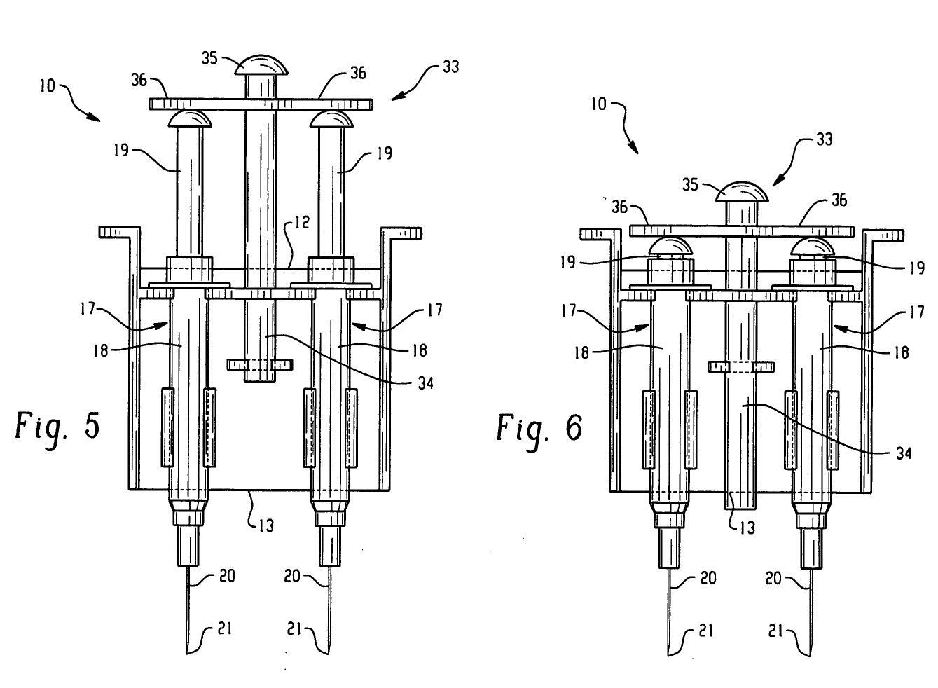

FIGS. 5 and 6 illustrate the use of the depressible push apparatus 33 to substantially simultaneously expel contents of the two syringes 17 through each of the injection needles 20 during injection. The needle tips 21 extend substantially an equal distance below the bottom edge 13 and the two injection needles 20 are spaced apart from each other at a distance that is at or within a two-point threshold of pain or pressure distance. If more than two syringes 17 are employed, each of the injection needles 20 is respectively spaced apart from its nearest neighbor by this same distance.

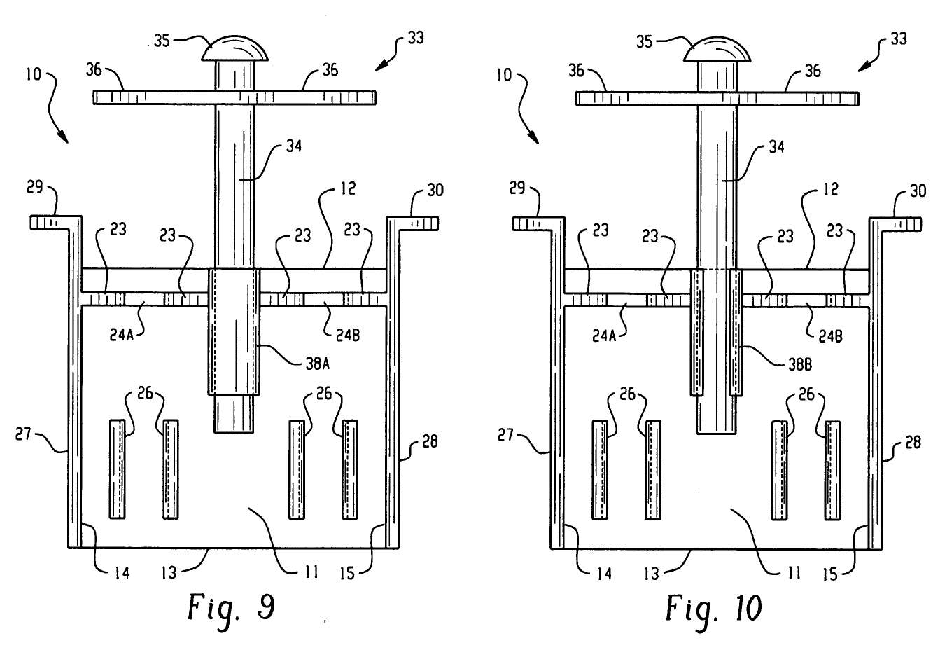

FIGS. 7 and 8 is a top view of an arrangement of the syringe holder 10 illustrating the depressible push apparatus 33 is both slideable and rotatable in a clockwise or counter clockwise direction, respectively, to allow clearance from the cross piece 36 for releasable attachment of the two syringes 17 to the syringe holder 10, especially when used with a stabilizing device that allows rotation of the plunger shaft such as, but not limited to, stabilizing devices 38A and/or 38B shown in FIGS. 9 and 10.

FIGS. 9 and 10 illustrate alternative arrangements of the syringe holder 10 in which the plunger shaft stabilizing device 38 comprises a closed tube 38A or an open tube 38B.

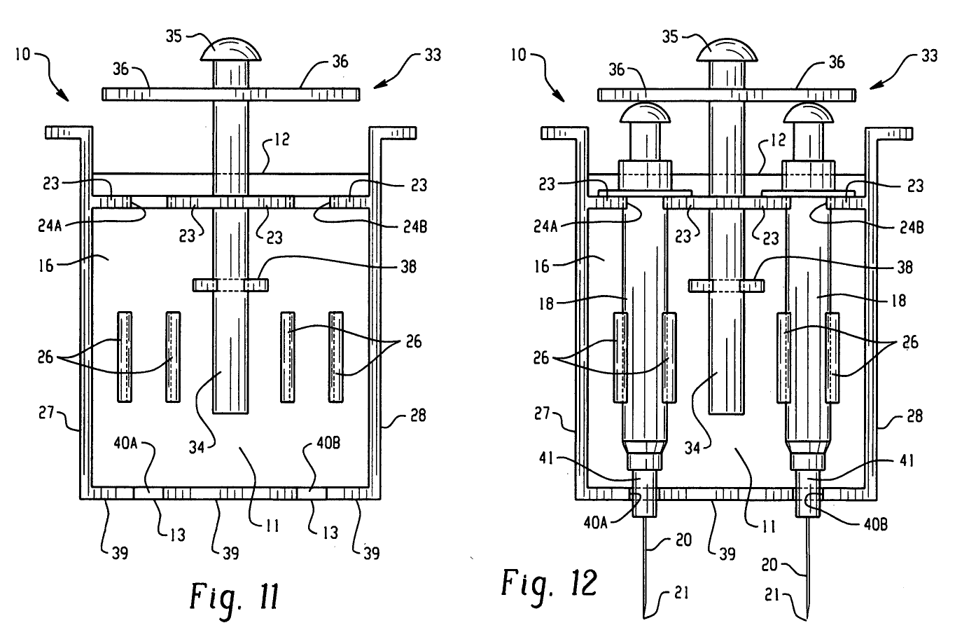

FIGS. 11 and 12 illustrate another arrangement of the syringe holder 10 that includes features according to any of FIGS. 1 through 10 and/or 13 through 16, respectively, but also includes a bottom wall 39 contiguous with the bottom edge 13 of the housing 11 and substantially perpendicular to the front surface 16 of the housing 11. The bottom wall 39 can be contiguous with one or both of the side walls 27, 28. The bottom wall 39 comprises a set of spaces 40A and 40B that are directly opposed to and aligned with the set of spaces 24A and 24B, respectively, between the set of ledges 23, and the respective stabilizing devices 26. The set of spaces 40A and 40B have a width sufficient to accommodate a syringe needle hub 41 but not sufficient to accommodate a syringe barrel 18.

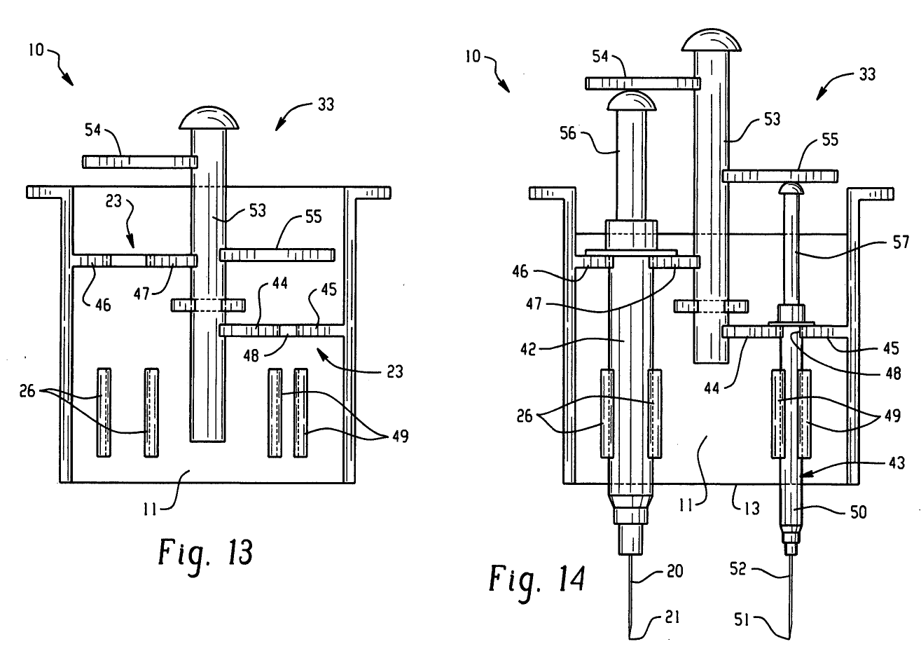

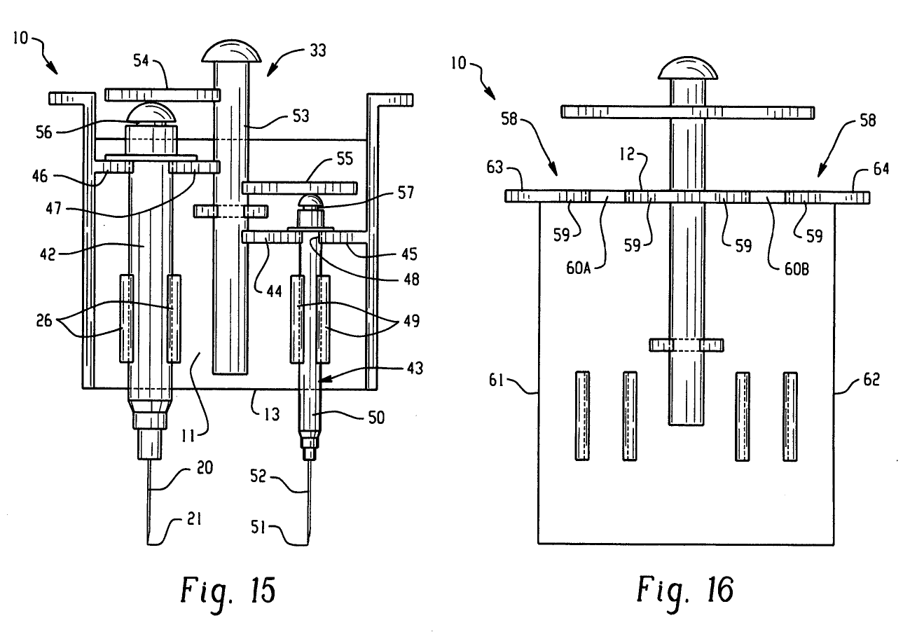

FIG. 13 illustrates another arrangement of the syringe holder 10 that accommodates two syringes having different sizes. As shown in FIGS. 14 and 15, syringe 42 is larger in length and diameter than syringe 43. To accommodate the smaller syringe 43 having a shorter length, two ledges 44, 45 of the set of ledges 23 are located below the ledges 46, 47 that accommodate the longer syringe 42. Both the space 48 between the two ledges 44, 45 and the flexible stabilizing device 49 aligned with the space 48 are narrow, to accommodate the smaller diameter syringe barrel 50 of syringe 43. The two ledges 44, 45 are positioned such that the needle tip 51 of the injection needle 52 of the smaller syringe 43 extends substantially an equal distance below the bottom edge 13 of the housing 11 as the needle tip 21 of the injection needle 20 of the larger syringe 42. The two injection needles 52 and 20 are spaced apart from each other at a distance that is at or within a two-point threshold of pain or pressure distance.

The cross piece of the plunger shaft 53 of the depressible push apparatus 33, illustrated in FIGS. 13, 14 and 15, comprises a first lateral cross piece section 54 and a second lateral cross piece section 55, offset from each other along the plunger shaft 53 such that the first cross piece section 54 is in contact with the syringe plunger 56 of syringe 42 at the same time as the second cross piece section 55 is in contact with the syringe plunger 57 of the syringe 43. FIGS. 14 and 15 illustrate the use of the depressible push apparatus 33 to substantially simultaneously expel contents of the two syringes 42, 43 through each of the respective injection needles 20 and 52 during injection.

FIG. 16 illustrates another arrangement of the syringe holder 10 in which the top edge 12 has a contiguous top wall 58 that comprises the set of substantially horizontal adjacent ledges 59 linearly separated by a set of spaces 60A, 60B. The set of adjacent ledges 59 are contiguous with the front surface 16 of the housing. In this arrangement, side walls may or may not be present. The top wall 58 can extend laterally beyond at least one of the side edges 61, 62 to provide one or more grip surfaces 63, 64 for ease of handling by the administrator of the injections. All other elements of this arrangement are the same as those shown in any of the previous figures.

In each of the arrangements illustrated in the Figures above, various elements of the syringe holder are interchangeable, without limitation. As a non-limiting example, the position of the top edge and/or the ledges can be varied; the presence of the bottom wall is optional; the grip surfaces can be formed by an extension of a top wall or a side wall, if present; the stabilizing devices can vary in number and shape; the depressible push apparatus can vary in size, shape and position; and the cross piece can comprise more than one section. Although the Figures illustrate the use of the syringe holder with two syringes, more than two parallel syringes can be accommodated by increasing the width of the housing and by adding the respective elements associated with containment of the additional syringe(s).

The syringe holder described above is light weight and can be inexpensively produced and all elements, excluding the depressible push apparatus, can be produced as a single molded piece, from a thermoplastic or thermoset plastic material. Similarly, the depressible push apparatus can be molded as a single piece and separately fitted to the syringe holder. The syringe holder, including or excluding the depressible push apparatus, can be disposable or can be reused. It can be easily cleaned in soapy water or gas sterilized, such as by hydrogen peroxide vapor or ethylene oxide gas. If sterilization by autoclave is desired, it is preferred that a thermoset material or a lightweight glass be employed.

This written description sets forth the best mode of carrying out the invention, and describes the invention so as to enable a person of ordinary skill in the art to make and use the invention, by presenting examples of the elements recited in the claims. The patentable scope of the invention is defined by the claims, and may include other examples that occur to those skilled in the art. Such other examples, which may be available either before or after the application filing date, are intended to be within the scope of the claims if they have elements that do not differ from the literal language of the claims, or if they have elements with insubstantial differences from the literal language of the claims.

I claim:

1. A syringe holder for multiple injections, comprising:
(a) a substantially flat housing comprising a top edge, a bottom edge, two side edges and a front surface, wherein the housing has a width sufficient to accommodate two or more substantially parallel syringes spaced apart from each other in a plane parallel to the front surface, wherein each syringe comprises a syringe barrel, a syringe plunger and an injection needle;
(b) a first set of substantially horizontal adjacent ledges contiguous with the front surface of the housing, wherein the first set of adjacent ledges is linearly separated by a set of first spaces, each of the first spaces being of sufficient width to accommodate the syringe barrel but not of sufficient width to accommodate a syringe lip contiguous with the syringe barrel, and wherein the first set of adjacent ledges is optionally contiguous with at least a portion of the top edge of the housing;
(c) a plurality of flexible stabilizing devices contiguous with the front surface of the housing and aligned with the set of first spaces for releasably securing the two or more substantially parallel syringes to the housing, wherein the injection needles comprise needle tips extending substantially an equal distance below the bottom edge of the housing, and wherein at least two of the injection needles are spaced apart from each other at a distance that is at or within a two-point threshold of pain or pressure distance;
(d) a depressible push apparatus comprising a plunger shaft and an associated cross piece, wherein the cross piece is configured to be in alignment with the two or more syringe plungers for substantially simultaneous depression of the syringe plungers; and
(e) a plunger shaft stabilizing device contiguous with the front surface of the housing for slideably engaging the plunger shaft.

2. The syringe holder of claim 1, wherein each of the first set of adjacent ledges further comprises a ledge wall for containing the syringe lip within a first space between two adjacent ledges.

3. The syringe holder of claim 1, wherein the top edge of the housing further comprises a contiguous lateral extension comprising a top wall that comprises a grip surface.

4. The syringe holder of claim 1, wherein the housing further comprises a side wall contiguous with the housing at a side edge.

5. The syringe holder of claim 4, wherein the side wall comprises a grip surface.

6. The syringe holder of claim 4, wherein the side wall comprises a contiguous lateral extension that comprises a grip surface.

7. The syringe holder of claim 1, wherein the housing further comprises a bottom wall contiguous with the bottom edge of the housing

8. The syringe holder of claim 7, wherein the bottom wall comprises a set of second spaces, each of the second spaces being in direct opposition to a corresponding first space of the first set of substantially horizontal adjacent ledges, each of the second spaces being of sufficient width to accommodate a syringe needle hub but not of sufficient width to accommodate the syringe barrel.

9. The syringe holder of claim 1, wherein the cross piece associated with the plunger shaft comprises a first lateral cross piece section and a second lateral cross piece section, wherein the cross piece sections are offset from each other along the plunger shaft.

10. The syringe holder of claim 1, wherein the cross piece sections are contiguous with the plunger shaft.

11. The syringe holder of claim 1, wherein the plunger shaft stabilizing device rotationally engages the plunger shaft.

12. The syringe holder of claim 1, wherein the multiple injections are intramuscular injections.
